(19) United States
(12) Patent Application Publication
(10) Pub. No.: US 2016/0317728 A1
(43) Pub. Date: Nov. 3, 2016

(54) MILK STORAGE BAG ADAPTER AND HOLDER

(71) Applicant: Evenflo Feeding Inc., West Chester, OH (US)

(72) Inventors: Frank Lewis, Fairfield, OH (US); Caitlin Knobbe, Loveland, OH (US)

(73) Assignee: Evenflo Feeding Inc., West Chester, OH (US)

(21) Appl. No.: 14/701,820

(22) Filed: May 1, 2015

(51) Int. Cl. A61M 1/06

(52) U.S. Cl. CPC A61M 1/062; A61M 1/06

(57) ABSTRACT

The present disclosure provides an adapter for use with a breast pump assembly for use for securely attaching a milk storage bag thereto. Specifically, the present disclosure provides an adapter having clips thereon for securely engaging multiple different kinds of breast milk storage bags securely thereto for the receiving of expressed breast milk therein.

### TECHNICAL FIELD

The present disclosure relates to an adapter for use with a breast pump assembly and with a storage bag for breast milk, and more particularly to such an adapter capable of being used with such storage bags.

### BACKGROUND OF THE DISCLOSURE

It is known to provide storage bags for breast milk that are adapted to be attached to breast pumps. For example, U.S. Pat. No. 6,575,202 to Lafond discloses a disposable sterile plastic bag for receiving and storing breast milk directly from the breast pump. The Lafond bag includes a liquid receiving chamber which can be accessed upon removal of a tear off strip. The Lafond bag also includes a closure member to close the bag once filled with milk. Once opened, the Lafond bag can be attached to an outlet port of a breast pump such that pumped milk is conveyed directly and sterilely into the bag for cold storage. The attachment mechanism disclosed in the '202 patent comprises a pair of metal strips or wires and a pair of adhesive tapes disposed outwardly over the metal wires to attach the metal wires to the front and rear sheets of the bag.

Similarly, U.S. Pat. No. 8,357,116 to Simdon discloses a bag attachment device for breast pumps that allows for the attachment of a breast milk storage bag to a breast pump through the use of an adapter. Specifically, the '116 patent discloses an adapter for use in connection with a beast pump assembly having mounting ears upon which a milk storage bag having apertures therein may be hung to suspend the bag in place below the breast pump assembly. The adapter is used to connect the milk storage bag to the breast pump assembly such that expressed milk from the breast pump horn is directly collected by the bag without need to connect a bottle to the breast pump assembly. The adapter disclosed in the '116 patent includes “ears” on either side thereof and the milk storage bag is provided with apertures therein to be received by the ears thus allowing the bag to hang beneath the adapter.

While the bag disclosed in the '202 patent and the adapter and bag disclosed in the '116 patent each have utility for their intended purposes, they do not resolve all of the issues involved with using milk storage bags in connection with otherwise standard breast pumps or breast pump assemblies. For example, while the bag disclosed in the '202 patent may be useful in some circumstances, in situations where that bag is not available, a user thereof has no alternate choice (such as a standard Ziploc® bag) that may be used securely, safely and in a sanitary manner to receive the expressed milk and then transfer the bag directly to cold storage. Further, with respect to the milk storage bag disclosed in the '116 patent, in situations where such a bag may not be readily available to a user thereof, the user has no alternate choice (such as a standard Ziploc® bag) that may be used securely, safely and in a sanitary manner.

Accordingly, it is desired to have an adapter for use in securely holding a milk storage bag below a collection tube for a breast pump assembly that securely holds the milk storage bag and allows for minimal movement thereof. It is further desired to have an adapter for use in connection with a milk storage bag that may be used with storage bags of many different configurations including otherwise standard Ziploc®-style storage bags. It is further desired to provide an extender for such an adapter for breast pump assemblies wherein additional extension is needed in order to securely hold the milk storage bag below the collection portion of the breast pump assembly.

### SUMMARY

The present disclosure provides an adapter for use in connection with a breast pump assembly and a milk storage bag that holds and provides support for the milk storage bag when it is in use allowing breast milk to be expressed directly into the milk storage bag.

More specifically, the present provides an adapter having clips thereon for securely engaging multiple different kinds of breast milk storage bags securely thereto for the receiving of expressed breast milk therein. In accordance with aspects of the disclosure, the milk storage bags may or may not include apertures on any upper portion thereof and the clips may be resiliently biased and include teeth on a lower portion thereof to engage corresponding teeth on the adapter. Also in accordance with some embodiments of the disclosure, an extension connector may be provided for extending the distance between the adapter and the breast pump assembly to provide additional clearance if necessary and/or desired.

### BRIEF DESCRIPTION OF THE DRAWINGS

Having thus generally described the nature of the disclosure, reference will now be made to the accompanying drawings, showing by way of illustration embodiments thereof, and in which:

FIG. 1 is a side elevational view of an adapter having bag storage clamps thereon for a breast pump assembly in accordance with at least one embodiment of the present disclosure;

FIG. 2 is a top perspective view of the adapter of FIG. 1;

FIG. 3 is a milk storage bag operable for use in connection with the adapter of FIG. 1 in accordance with at least one embodiment of the present disclosure;

FIG. 4 is a top perspective view of the adapter of FIG. 1 engaged with the milk storage bag of FIG. 3;

FIG. 5 is a side elevational view of the adapter of FIG. 1 attached to a breast pump assembly showing the bag storage clamps in a closed configuration in accordance with aspects of the present disclosure; and

FIG. 6 is a side elevational view of the adapter of FIG. 1 attached to an extension connector attached to a breast pump assembly showing the bag storage clamps in an open configuration in accordance with at least one embodiment of the disclosure

### DETAILED DESCRIPTION

While an adapter for use in connection with a breast pump assembly according to this disclosure may be embodied in many forms, there is shown in the drawings and will herein be described in detail one or more embodiments of such an adapter, as well as related extension connector and milk storage bag, with the understanding that this disclosure is to be considered an exemplification of the principles disclosed herein and is not intended to be limited to merely the illustrated and discussed embodiments.

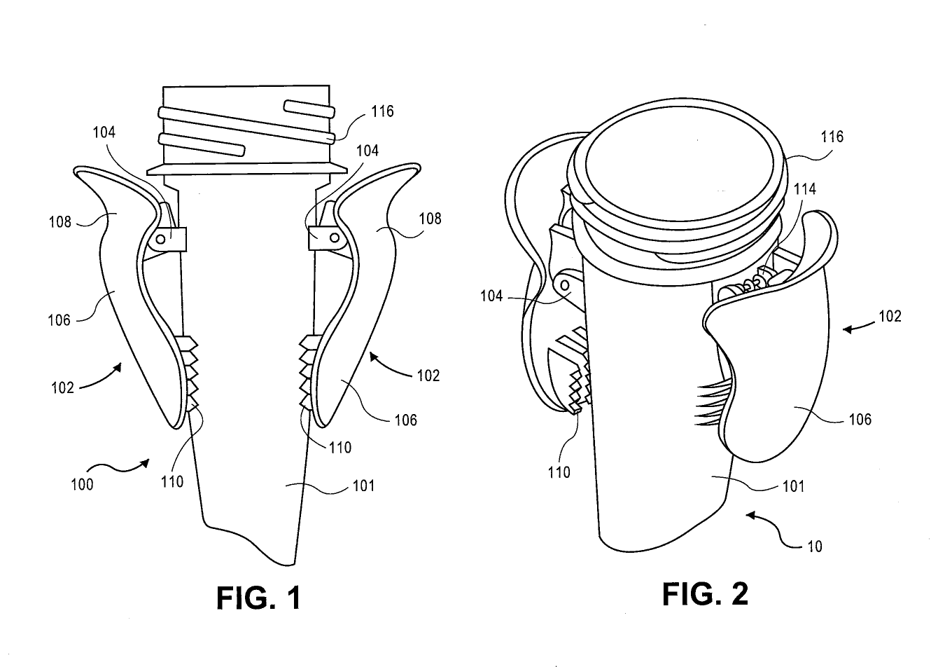

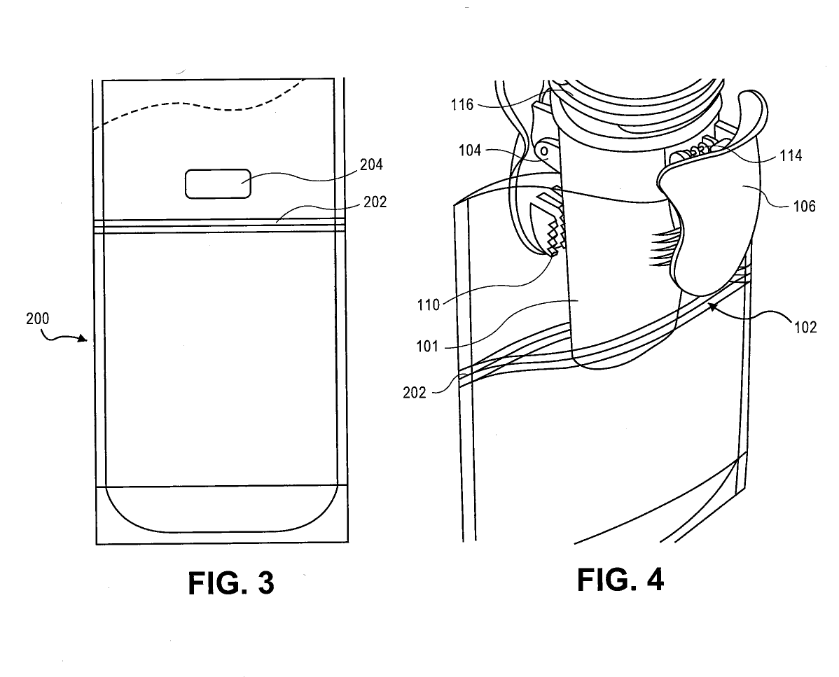

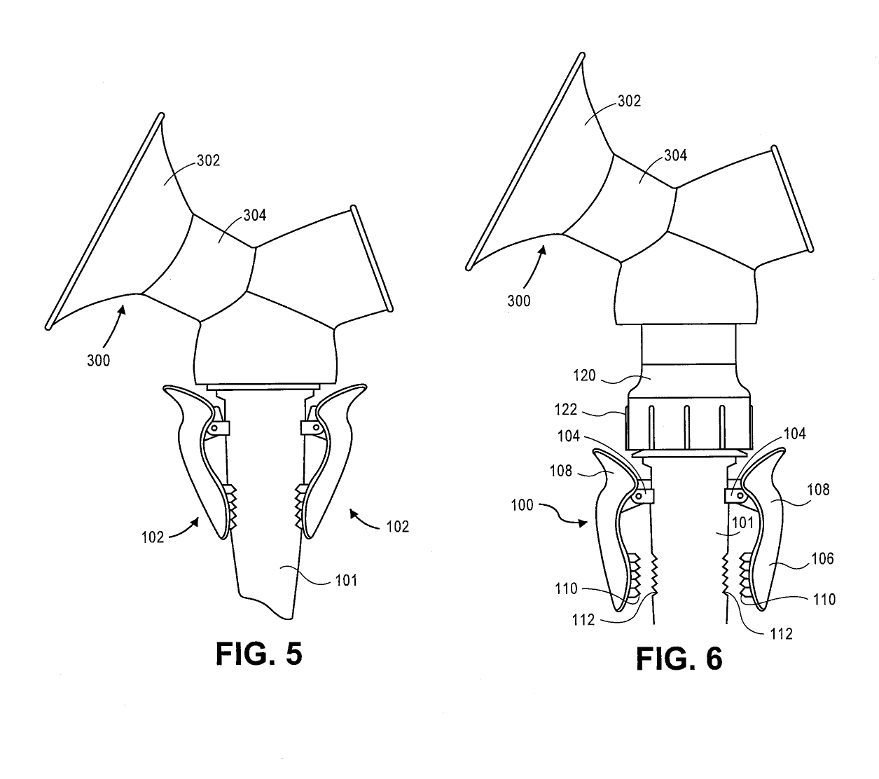

In this regard, and specifically with reference to accompanying drawing FIGS. 1-6, in accordance with embodiments of the disclosure, an adapter 100 is provided for connecting a milk storage bag 200 to a breast pump assembly 300. The adapter 100 may comprise any desired shape and configuration operable to convey breast milk from the breast pump assembly 300 to the milk storage bag 200 as would be understood by those of ordinary skill in the art. The adapter 100 may be provided with at least one, but preferably two, attachment mechanisms. The attachment mechanisms may be mounted on the adapter surface on opposite sides thereof. In accordance with the embodiment of the disclosure shown in the drawings, the attachment mechanisms may specifically comprise clips 102.

In accordance with aspects of the present disclosure, the clips 102 may be mounted on the adapter 100 by hinges 104. The clips 102 may be comprised of levers 106 cantilevered on the hinges 104 and may include thumb portions 108 on one end thereof to facilitate operation by a user and teeth 110 on the opposite end thereof to facilitate the clamping and holding of the milk storage bag 200. As best shown in FIG. 6, corresponding teeth 112 may be provided on the adapter 100 to facilitate engagement of the teeth 110 thereby aiding in more securely clamping a portion of the milk storage bag 200 therebetween.

In accordance with an embodiment of the disclosure, the clips 102 are preferably resiliently biased against the funnel portion 101 of the adapter. In accordance therewith, springs 114 may be provided on the hinges 104 to resiliently bias the teeth 110 on the lower portion of the levers 106 against the funnel portion 101 of the adapter 100, and more particularly, to engage the corresponding teeth 112 formed on the adapter 100. As will be understood by those of ordinary skill in the art, the strength of springs 114 may be varied to provide more or less clamping force, as desired or necessary, for the levers 106. Furthermore, while a torsion spring is shown in the drawings, alternate springs and/or resiliently biasing mechanisms (such as leaf springs) may be used in accordance herewith and are considered within the purview of the disclosure.

The adapter 100 may be provided with threads 116 on an upper portion thereof for releasably connecting the adapter 100 to corresponding threads provided on the breast pump assembly 300. In one embodiment of the disclosure, the threads 116 may be the same size and shape as standard threads generally provided on nursers. In accordance with some embodiments of the disclosure, the milk storage bag 200 may include a recloseable seal 202 and mounting apertures 204 therein.

In accordance with one aspect of the disclosure, as best seen in FIG. 6, an extension connector 120 may be provided for allowing additional clearance, if desired, between the adapter 100 and the breast pump assembly 300. The extension connector 120 may include female threads on an interior portion thereof (not shown) to engage corresponding threads 116 on the adapter and may include threads (not shown) to engage corresponding threads (not shown) on the breast pump assembly 300. The extension connector 120 may also have a gripping portion 122 on an outside portion thereof to facilitate gripping of the extension connector 120 by a user thereof.

In accordance with the disclosure, the breast pump assembly 300 may include a funnel-shaped breast shield 302 that is sized and shaped for engagement with at least a portion of a breast as is known in the art. The funnel shape breast shield 302 may lead to a conduit 304 for providing a pathway for expressed breast milk to be directed to through the adapter 100 (and/or extension connector 120) to the milk storage bag 200 held by clips 102.

In accordance with the disclosure, the milk storage bag 200 receives and stores breast milk pumped from the breast pump assembly 300. The bag may be of any desired shape and/or size may include otherwise standard zip-type storage bags. Indeed, a number of known designs exist for “male/female” zip-type closures, any and/or all of which are considered operable within aspects of the present disclosure and are included within the purview thereof. In accordance with one aspect of the disclosure, the milk storage bag 200 may be provided with apertures 204 on an upper portion thereof to receive at least a portion of the teeth 110 on the levers 106 of the clips 102 therethrough to facilitate in the gripping and holding of the milk storage bag 200. Conversely, it is completely operable and within the scope of the disclosure to utilize milk storage bags 200 that have no apertures on the upper portions thereof. In accordance with such an embodiment, the springs 114 are strong enough that the clamping force of the clips 102 on the top portion of the milk storage bag 200 is sufficient to securely hold the milk storage bag 200 even when considering the additional weight of the expressed breast milk (not shown).

Milk storage bag 200 may further include a label area (not shown) located on the outer side thereof to allow for a user thereof to mark the bag with information about the contents, such as, for example, the date and/or time the breast milk was collected. Calibrated markings (not shown) as for milk volume may additionally be provided if desired. In an embodiment of the disclosure, the length of the funnel portion 101 of the adapter 100 may be selected such that it is long enough to extend into the milk storage bag 200 sufficiently past the recloseable seal 202 so as to prevent milk from splashing on the recloseable seal 202 in use.

In operation, the adapter 100, and extension connector 120 (if desired) is attached to the breast pump assembly 300 using the threaded connections. A milk storage bag 200 is then clamped by the clips 102 through actuation of the thumb portions 108 on the levers 106 securely in place. In accordance with an embodiment wherein apertures 204 are employed on the milk storage bag 200, the user may preferably align the apertures 204 such that at least a portion of the teeth 110 on the levers 106 proceed through the apertures 204 to directly engage the corresponding teeth 112 provided on the lower portion of the adapter 100.

The many features and advantages of the disclosure are apparent from the detailed specification, and, thus, it is intended by the appended claims to cover all such features and advantages of the disclosure which fall within the true spirit and scope of the disclosure. Further, since numerous modifications and variations will readily occur to those skilled in the art, it is not desired to limit the disclosure to the exact construction and operation illustrated and described, and, accordingly, all suitable modifications and equivalents may be resorted to that fall within the scope of the disclosure.

What is claimed is:

1. A milk storage bag adapter operable to convey breast milk from a breast pump assembly to a milk storage bag comprising:
an adapter having a funnel portion and a threaded top portion;
at least one clip attached to the adapter, the clip having teeth thereon for engaging a milk storage bag, wherein the clip is mounted on the adapter such that the teeth are resiliently biased against the funnel portion of the adapter.

2. The milk storage bag adapter of claim 1 wherein the adapter is provided with two clips.

3. The milk storage bag adapter of claim 2 wherein the clips are comprised of levers cantilevered by hinges to the adapter.

4. The milk storage bag adapter of claim 3 wherein the clips are resiliently biased by springs.

5. The milk storage bag adapter of claim 4 wherein said springs comprise torsion springs.

6. The milk storage bag adapter of claim 1 wherein the clip includes a thumb portion thereon.

7. The milk storage bag adapter of claim 1 wherein the funnel portion includes teeth thereon for engage the corresponding teeth located on the clip.

8. The milk storage bag adapter of claim 1 further comprising an extension connector shaped to threadably engage the threaded top portion of the adapter on one end thereof and including threads on an opposite end thereof.

9. A breast pump assembly comprising:
a funnel-shaped breast shield that is sized and shaped for engagement with at least a portion of a breast;
a conduit for providing a pathway for expressed breast milk the conduit having threads on a lower portion thereof;
an adapter having a funnel portion and a threaded top portion for engaging the threads on the conduit;
at least one clip attached to the adapter, the clip having teeth thereon, wherein the clip is mounted on the adapter such that the teeth are resiliently biased against the funnel portion of the adapter; and
a milk storage bag having an upper portion capable of being securely clamped between the teeth on the clip and the funnel portion of the adapter for receiving breast milk therein.

10. The breast pump assembly of claim 9 wherein the adapter is provided with two clips.

11. The breast pump assembly of claim 10 wherein the clips are comprised of levers cantilevered by hinges to the adapter.

12. The breast pump assembly of claim 11 wherein the clips are resiliently biased by springs.

13. The breast pump assembly of claim 12 wherein said springs comprise torsion springs.

14. The breast pump assembly of claim 9 wherein the clip includes a thumb portion thereon.

15. The breast pump assembly of claim 9 wherein the funnel portion includes teeth thereon for engage the corresponding teeth located on the clip.

16. The breast pump assembly of claim 9 further comprising an extension connector shaped to threadably engage the threaded top portion of the adapter on one end thereof and including threads on an opposite end thereof.

17. The breast pump assembly of claim 9 wherein the milk storage bag includes a recloseable seal located proximal the upper portion thereof.

18. The breast pump assembly of claim 17 wherein the funnel portion protrudes into the milk storage bag past the recloseable seal when the milk storage bag is clamped thereto

19. A breast pump assembly comprising:
a funnel-shaped breast shield that is sized and shaped for engagement with at least a portion of a breast;
a conduit for providing a pathway for expressed breast milk the conduit having threads on a lower portion thereof;
an adapter having a funnel portion and a threaded top portion for engaging the threads on the conduit;
two clips attached to the adapter, the clips having teeth thereon, wherein the clips are mounted on the adapter such that the teeth are resiliently biased against the funnel portion of the adapter.

20. The breast pump assembly of claim 19 further comprising a milk storage bag having an upper portion capable of being securely clamped between the teeth on the clips and the funnel portion of the adapter for receiving breast milk therein.
